(19) United States
(12) Patent Application Publication
(10) Pub. No.: US 2011/0142965 A1
(43) Pub. Date: Jun. 16, 2011

(54) SKIN MOISTURIZER AND WASHING FORMULATION

(76) Inventor: Bob Walke, Flower Mound, TX (US)

(21) Appl. No.: 12/965,467

(22) Filed: Dec. 10, 2010

Related U.S. Application Data
(60) Provisional application No. 61/285,452, filed on Dec. 10, 2009.

(51) Int. Cl. A61K 36/185; A61Q 19/00

(52) U.S. Cl. 424/725

(57) ABSTRACT

Disclosed is a liquid skin cleansing and moisturizing composition comprising a skin moisturizing agent, a cationic guar gum, an anionic surfactant, a zwitterionic surfactant, and a non-lipid structuring agent, wherein the composition is dermatologically acceptable and formulated as a rinse-off composition.

### CROSS REFERENCE TO RELATED APPLICATIONS

This application claims the benefit of U.S. Provisional Application No. 61/285,452, filed Dec. 10, 2009. The contents of this referenced application are incorporated by reference.

### BACKGROUND OF THE INVENTION

A. Field of the Invention

The present invention relates generally to skin cleansing compositions that can also moisturize skin.

B. Description of Related Art

Skin cleansing compositions are typically applied to skin and rinsed-off with water (e.g., rinse-off product). Some problems associated with skin cleansing compositions include skin irritation and dry skin. Typical skin moisturizing compositions are topically applied to skin with the intent that they remains on the skin for an extended period of time (e.g., a leave-on product).

Although some dual action cleansing/moisturizing rinse-off products exist, such products tend to be unstable, lack the ability to effectively moisturize the skin, and can cause skin irritation. Further, many of the products leave the skin feeling sticky.

### SUMMARY OF THE INVENTION

The present invention overcomes deficiencies in the art by providing stable cleansing compositions that can also effectively moisturize the skin. In one aspect, there is disclosed a rinse-off skin cleansing and moisturizing composition that includes a skin moisturizing agent, a cationic guar gum, an anionic surfactant, a zwitterionic surfactant, and a non-lipid structuring agent, wherein the composition is dermatologically acceptable and formulated as a rinse-off composition. In one embodiment, the composition includes 15 to 30% w/w of the skin moisturizing (or 16, 17, 18, 19, 20, 21, 22, 23, 24, 25, 26, 27, 28, or 29% of the moisturizing agent or any integer or range therein), 0.1 to 1% w/w of the cationic guar gum is guar hydroxypropyltrimonium chloride (or 0.2, 0.3, 0.4, 0.5, 0.6, 0.7, 0.8, or 0.9% of the cationic guar gum or any integer or range therein), 10 to 25% w/w of the anionic surfactant (or 11, 12, 13, 14, 15, 16, 17, 18, 19, 20, 21, 22, 23, or 24% of the anionic surfactant or any integer or range therein), 3 to 7% w/w of the zwitterionic surfactant (or 4, 5, or 6% of the zwitterionic surfactant or any integer or range therein, and 3 to 7% w/w of the non-lipid structuring agent (or 4, 5, or 6% of the non-lipid structuring agent, or any integer or range therein). In one aspect, the skin moisturizing agent is mineral oil or petrolatum or a combination thereof, the cationic guar gum is guar hydroxypropyltrimonium choloride, the anionic surfactant is selected from the group consisting of sodium laureth sulfate, sodium laureth-5 sulfate, sodium laureth-7 sulfate, sodium laureth-8 sulfate, sodium laureth-12 sulfate, sodium laureth-40 sulfate, sodium lauryl sulfate, and any combination thereof, the zwitterionic surfactant is cocamidopropyl hydroxysultaine, and the non-lipid structuring agent is an acrylates copolymer. In one embodiment, the composition includes 15 to 20% w/w of mineral oil or petrolatum or a combination thereof, 0.1 to 0.5% w/w of guar hydroxypropyltrimonium choloride, 14 to 20% w/w of sodium laureth sulfate, 3 to 7% w/w of cocamidopropyl hydroxysultaine, and 3 to 7% w/w of an acrylates copolymer. In another aspect, the composition can further include, consist essentially of, or consist of, any one of, any combination of, or all of the following ingredients: water; EDTA; citric acid; sodium chloride; lauric acid; glycerin; PEG-120 methyl glucose dioleate; triethanolamine; DMDM hydantoin; shea butter; tocopheryl acetate; a fragrance; and/or a preservative. In a particular aspect, the composition further includes less than 50% w/w of water, 0.1 to 0.5% w/w of EDTA, 0.001 to 0.01% w/w of citric acid, 0.001 to 0.01% w/w of sodium chloride, 1 to 5% w/w of lauric acid, 1 to 5% w/w of glycerin, 0.1 to 1% w/w of PEG-120 methyl glucose dioleate, 1 to 5% w/w of triethanolamine, 0.1 to 1% w/w of DMDM hydantoin, 0.1 to 5% w/w of shea butter; 0.1% to 5% of tocopheryl acetate; 1 to 5% w/w of a fragrance, and/or 0.1 to 1% w/w of a preservative. In another aspect, the liquid cleansing and moisturizing composition includes, consists essentially of, or consists of the ingredients and percentages of the ingredients identified in Tables 1 or 2 of this specification, which are incorporated into this section by reference. In another aspect, the liquid skin cleansing and moisturizing composition does not include petrolatum, ammonium laureth sulfate, ammonium lauryl sulfate, sodium lauryoamphoacetate, trihydroxystearin, sodium benzoate, niacinamide, PEG-14, retinyl palmitate, glycine soja oil, sunflower seed oil, cocamidopropyl betaine, cocamide MEA, polybutene, polyisobutene, isostearic acid, PEG-5 cocamide, acrylates/beheneth-25 methacrylate copolymer, PEG-30 dipolyhydroxystearate, tetrasodium EDTA, etidronic acid, iodopropynyl butylcarbamate, titanium dioxide, hydrogenated polyisobutene, sodium lauryoyl sarcosinate, sodium hydroxypropyl starch phosphate, lauryl alcohol, propylene glycol, and/or cyamopsis tetragonoloba (guar) gum. In one aspect, the composition does not include a lamellar phase. In one aspect, the composition does not include a crystalline hydroxy-containing stabilizer or a tri-hydroxystearin. In one aspect, the composition does not include a crystalline hydroxy-containing stabilizer and a tri-hydroxystearin. In one aspect, the liquid skin cleansing and moisturizing composition includes an aqueous phase and an oil phase. In one aspect, the composition does not include a silicone containing compound (e.g., cyclomethicone, dimethicone, etc.). The oil phase can include, consist essentially of, or consist of the skin moisturizing agent (e.g., mineral oil or petrolatum or a combination thereof). The oil phase can include at least 85, 86, 87, 88, 89, 90, 91, 92, 93, 94, 95, 96, 97, 98, 99% or more of the skin moisturizing agent (e.g., mineral oil or petrolatum or a combination thereof). The liquid skin cleansing and moisturizing composition can have a viscosity less than 25,000 cps, as measured on a Brookfield Viscometer using a TC spindle at 2.5 rpm at 25° C., which can allow for the composition to be squeezed, pumped, or otherwise dispensed from a bottle. In one aspect, the composition has a viscosity of 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 21,000, 22,000, 23,000, 24,000, or 25,000 cps (or any integer or range therein), as measured on a Brookfield Viscometer using a TC spindle at 2.5 rpm at 25° C. The composition can include less than 50%, 45%, 40%, 35%, 30%, 25%, 20%, 15%, 10%, 5%, or less, w/w of water. In some instances, the composition is anhydrous or substantially anhydrous. In one aspect, the total amount of surfactants in the composition is between 15 and 30%, 15 and 25%, 15 and 20%, or less than 30, 29, 28, 27, 26, 25, 24, 23, 22, 21, 20, 19, 18, 17, 16, or 15% (or any integer or range therein).

In one embodiment there is disclosed a liquid skin cleansing and moisturizing composition comprising a hydrophobic phase comprising petrolatum in an amount of at least 20, 30, 40, 50, 60, 70, 80, 90, 95, or 100% by weight of the hydrophobic phase, a hydrophilic phase comprising water, a cationic guar gum, and a non-lipid structuring agent, and a combination of anionic and zwitterionic surfactants, wherein the total amount of water in the composition is less than 70, 60, 50, 40, 30, 20, 10, 5, 1% by weight of the composition, wherein the total amount of surfactants within the composition is between 5 and 50%, 10 and 40%, 15 and 25%, or 18 and 22% by weight of the composition, wherein the total amount of the non-lipid structuring agent within the composition is between 1 and 10%, 3 and 7%, or 4 and 6% by weight of the composition, wherein the composition is in liquid form and has a viscosity range between 5,000 cps and 50,000 cps, 10,000 and 40,000 cps, or 15,000 to 25,000 cps, as measured on a Brookfield Viscometer using a TC spindle at 2.5 rpm at 25° C., wherein the composition does not include a crystalline hydroxy-containing stabilizer and a tri-hydroxystearin. In one aspect, the hydrophobic phase can consist of only petrolatum, shea butter extract, and/or tocopheryl acetate. In certain aspects, the composition does not include ammonium laureth sulfate, ammonium lauryl sulfate, sodium lauryoamphoacetate, trihydroxystearin, sodium benzoate, niacinamide, PEG-14, retinyl palmitate, glycine soja oil, sunflower seed oil, cocamidopropyl betaine, cocamide MEA, polybutene, polyisobutene, isostearic acid, PEG-5 cocamide, acrylates/beheneth-25 methacrylate copolymer, PEG-30 dipolyhydroxystearate, tetrasodium EDTA, etidronic acid, iodopropynyl butylcarbamate, titanium dioxide, hydrogenated polyisobutene, sodium lauryoyl sarcosinate, sodium hydroxypropyl starch phosphate, lauryl alcohol, propylene glycol, and cyamopsis tetragonoloba (guar) gum.

Also disclosed is a method of cleansing and moisturizing skin comprising applying any one of the compositions disclosed throughout this specification to skin followed by rinsing the composition from the skin. The composition can be rinsed with water. The composition can be rinsed within 1, 2, 3, 4, 5, 6, 7, 8, 9, 10, 15, 20, or 30 minutes after it has been applied to skin. The skin can be facial skin, arm skin, leg skin, neck skin, back skin, chest skin, etc.

The compositions in non-limiting aspects can have a pH of about 6 to about 9. In other aspects, the pH can be 1, 2, 3, 4, 5, 6, 7, 8, 9, 10, 11, 12, 13, or 14.

In other aspects, the compositions of the present invention can include any one of, any combination of, or all of other cosmetic or pharmaceutical ingredient described in this specification (e.g., UV absorbing agents, moisturizing agents, antioxidants, structuring agents, emulsifiers, silicone containing compounds, essential oils, thickening agents, and/or preservatives). The amounts of such ingredients can range from 0.0001 to 99% or any range or integer derivable therein as disclosed in this specification.

Also disclosed is a method of treating or preventing a skin condition comprising topical application of any one of the compositions disclosed in this specification to skin in need of such treatment. The method can include topical application of the composition to a portion of skin in need of such composition (e.g., skin having a skin condition), wherein topical application reduces or prevents the skin condition when compared to skin that has a skin condition and that has not been treated with the composition. Non-limiting examples of skin conditions include pruritus, spider veins, lentigo, age spots, senile purpura, keratosis, melasma, blotches, fine lines or wrinkles, nodules, sun damaged skin, dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, acne, impetigo, erysipelas, erythrasma, eczema, and other inflammatory skin conditions. In certain non-limiting aspects, the skin condition can be caused by exposure to UV light, age, irradiation, chronic sun exposure, environmental pollutants, air pollution, wind, cold, heat, chemicals, disease pathologies, smoking, or lack of nutrition. The skin can be facial skin or non-facial skin (e.g., arms, legs, hands, chest, back, feet, etc.). The method can further comprise identifying a person in need of skin treatment. The person can be a male or female. The age of the person can be at least 1, 2, 3, 4, 5, 6, 7, 8, 9, 10, 15, 20, 25, 30, 35, 40, 45, 50, 55, 60, 65, 70, 75, 80, 85, 90, 95, or more years old, or any range derivable therein. The method can also include topically applying an amount effective to: increase the stratum corneum turnover rate of the skin; increase collagen synthesis in fibroblasts; increase cellular anti-oxidant defense mechanisms (e.g., exogenous additions of anti-oxidants can bolster, replenish, or prevent the loss of cellular antioxidants such as catalase and glutathione in skin cells (e.g., keratinocytes, melanocytes, langerhans cells, etc.) which will reduce or prevent oxidative damage to the skin, cellular, proteins, and lipids); inhibit melanin production in melanocytes; reduce or prevent oxidative damage to skin (including reducing the amount lipid peroxides and/or protein oxidation in the skin).

Also disclosed is a method of lightening skin or evening skin tone comprising applying the compositions of the present invention to the skin. The method can further comprise identify a person in need of lightening skin or evening skin tone. The methods can further include inhibiting melanogenesis in a skin cell, inhibiting tyrosinase or tyrosinase synthesis in a skin cell, or inhibiting melanin transport to keratinocytes in a skin cell. The composition can act as an alpha melanin stimulatory hormone antagonist. The composition can even out pigmentation of the skin. In non-limiting aspect, lightening skin can include reducing the appearance of an age spot, a skin discoloration, or a freckle by topical application of the composition to skin having an age spot, skin discoloration, a freckle, etc.

Also disclosed is a method of treating hyperpigmentation comprising applying the compositions of the present invention to the skin. The method can also comprise identifying a person in need of treating hyperpigmentation. Additional methods contemplated by the inventor include methods for reducing the appearance of an age spot, a skin discoloration, or a freckle, reducing or preventing the appearance of fine lines or wrinkles in skin, or increasing the firmness of skin.

In a particular embodiment there is disclosed a method of reducing the appearance of a skin condition comprising topically applying any one of the compositions described in this specification to the skin condition, wherein topical application of the composition to skin condition reduces the appearance of the skin condition. The skin condition can be any one of those described in this specification. In particular aspects, the skin condition is a fine line or wrinkle, uneven skin tone, or an age spot. The skin condition can be located on facial skin, arm skin, leg skin, chest skin, abdomen skin, back skin etc.

In yet another embodiment there is disclosed a method of increasing the firmness of skin comprising topically applying any one of the compositions described in this specification to skin in need thereof (a non-limiting example of which can be sagging skin, aged skin, skin that has reduced elasticity, skin that has skin cells having inadequate amounts of collage, fibronectin, or laminin or all of such proteins, etc.), wherein topical application of the composition to skin increases the firmness of skin. The composition can be used on facial skin, arm skin, leg skin, chest skin, abdomen skin, back skin, etc.

In still another embodiment there is disclosed a method of increasing collagen, fibronectin, or laminin production in a skin cell comprising topically applying any one of the compositions disclosed in this specification to a skin cell that is in need of collagen, fibronectin, or laminin production, wherein the topical application of the composition to the skin cell increases collagen, fibronectin, or laminin production in the skin cell. Typical skin cells can be found in skin that is aged, damaged by UV radiation, damaged by environmental factors, wrinkles, etc. In certain aspects, collagen, fibronectin, and laminin production are increased in the skin cell.

Also contemplated are kits that includes the compositions of the present invention. In certain embodiments, the composition is comprised in a container. The container can be a bottle, dispenser, or package. The container can dispense a pre-determined amount of the composition. In certain aspects, the compositions is dispensed in a spray, dollop, or liquid. The container can include indicia on its surface. The indicia can be a word, an abbreviation, a picture, or a symbol.

The compositions and methods for their use can “comprise,” “consist essentially of,” or “consist of” any of the ingredients disclosed throughout the specification. “Consist essentially of” in the context of the present invention means that the compositions do not include additional ingredients that would materially affect the washing and moisturizing ability of the compositions of the present invention.

It is contemplated that any embodiment discussed in this specification can be implemented with respect to any method or composition of the invention, and vice versa. Furthermore, compositions of the invention can be used to achieve methods of the invention.

In one embodiment, compositions of the present invention can be pharmaceutically or cosmetically elegant. “Pharmaceutically elegant” and/or “cosmetically elegant” describes a composition that has particular tactile properties which feel pleasant on the skin (e.g., compositions that are not too watery or greasy, compositions that have a silky texture, compositions that are non-tacky or sticky, etc.). Pharmaceutically or cosmetically elegant can also relate to the creaminess or lubricity properties of the composition or to the moisture retaining properties of the composition.

“Topical application” means to apply or spread a composition onto the surface of keratinous tissue. “Topical skin composition” includes compositions suitable for topical application on keratinous tissue. Such compositions are typically dermatologically-acceptable in that they do not have undue toxicity, incompatibility, instability, allergic response, and the like, when applied to skin. Topical skin care compositions of the present invention can have a selected viscosity to avoid significant dripping or pooling after application to skin.

“Dermatologically acceptable carrier, vehicle, or medium” means a carrier, vehicle, or medium into which the active ingredients can be effectively incorporated into. A dermatologically acceptable carrier is design to reduce or avoid undue toxicity, incompatibility, instability, allergic response, and the like, when applied to skin. Further, the compositions can be dermatologically acceptable in that they reduce or avoid undue toxicity, incompatibility, instability, allergic response, and the like, when applied to skin.

“Keratinous tissue” includes keratin-containing layers disposed as the outermost protective covering of mammals and includes, but is not limited to, skin, hair and nails.

The terms “mixture,” “mix,” and “mixing” or any variants of these terms, when used in the claims and/or specification includes, stirring, blending, dispersing, milling, homogenizing, and other similar methods. The mixing of the components or ingredients of the disclosed compositions can form into a solution. In other embodiments, the mixtures may not form a solution. The ingredients/components can also exist as undissolved colloidal suspensions.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “treating,” “inhibiting,” or “reducing” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

In today's image conscious society, people are continually looking for a product that can effectively wash and moisturize the skin at the same time. The composition of the present invention provides for a dual action skin cleanser/skin moisturizer that includes a combination of the following ingredients that brings about this effect: (a) a skin moisturizing agent; (b) a cationic guar gum; (c) an anionic surfactant; (d) a zwitterionic surfactant; and (e) a non-lipid structuring agent. These and other aspect of the present invention are described in further detail below.

### A. Skin Moisturizing Agents

The skin moisturizing agent provides for the moisturizing effect of the composition. Non-limiting examples of skin moisturizing agents that can be used in the context of the present invention include those known by persons of ordinary skill in the art (see, e.g., International Cosmetic Ingredient Dictionary and Handbook, 12^{th} Ed. (2008), which is incorporated by reference). In particular embodiments, the moisturizing agent is petrolatum or an oil (e.g., mineral oil, paraffinic oils, napthenic oils, aromatic oils, essential oils, etc.). In particular aspects, the mineral oil can be heavy or light mineral oil. Mineral oil is commercially available from a wide variety of sources (see, e.g., International Cosmetic Ingredient Dictionary and Handbook, vol. 2, 12^{th} Ed., vol. 2, pages 1597-1600 (2008), which is incorporated by reference).

### B. Cationic Guar Gums

The cationic guar gum is used to help keep the skin moisturizing agent on the skin after the composition has been rinsed from the skin. In one aspect, the cationic guar gum can adhere to the skin while also adhering to the moisturizing agent (e.g., mineral oil). Non-limiting examples of cationic guar gums that can be used in the context of the present invention include those known by persons of ordinary skill in the art (see, e.g., International Cosmetic Ingredient Dictionary and Handbook, 12^{th} Ed. (2008), which is incorporated by reference). In one aspect, the cationic guar gum can be guar hydroxypropyltrimonium chloride, which is a water-soluble quaternary ammonium derivative of natural guar gum. It is commercially available from a wide variety of sources (e.g., DeWolf Chemical Inc., Rhode Island (U.S.A.).

### C. Anionic Surfactants

The anionic surfactant is used as a cleansing agent by attaching to dirt and oily substances on the skin and removing the same once the composition is rinsed from the skin. Non-limiting examples of anionic surfactants that can be used in the context of the present invention include those known by persons of ordinary skill in the art (see, e.g., International Cosmetic Ingredient Dictionary and Handbook, 12^{th} Ed. (2008), which is incorporated by reference). Non-limiting examples include: sodium laurylsulfate, sodium myristylsulfate and sodium palmitylsulfate as the alkylsulfates; sodium lauryl ether sulfate, sodium myristyl ether sulfate and sodium palmityl ether sulfate as the alkyl ether sulfates; sodium salts, potassium salts, triethanolamine salts or arginine salts of lauric acid, myristic acid, palmitic acid or stearic acid as the fatty acid soaps; sodium salts, potassium salts, triethanolamine salts or arginine salts of monolaurylphosphoric acid as the monoalkyl phosphates; sodium salts, potassium salts, triethanolamine salts or arginine salts of cocoylglutamic acid, lauroylglutamic acid or myristoylglutamic acid as the acyl L-glutamates; and sodium salts, potassium salts, triethanolamine salts or arginine salts of cocoylisethionic acid or lauroylisethionic acid as the acyl isethionates. In particular aspect, the anionic surfactant is selected from the group consisting of sodium laureth sulfate, sodium laureth-5 sulfate, sodium laureth-7 sulfate, sodium laureth-8 sulfate, sodium laureth-12 sulfate, sodium laureth-40 sulfate, sodium lauryl sulfate, and any combination thereof. Anionic surfactants such as those described in the specification are commercially available from a wide variety of sources (see, e.g., International Cosmetic Ingredient Dictionary and Handbook, 12^{th} Ed. (2008), which is incorporated by reference).

### D. Zwitterionic Surfactants

The zwitterionic surfactant is also used as a cleansing agent by attaching to dirt and oily substances on the skin and removing the same once the composition is rinsed from the skin. Non-limiting examples of zwitterionic surfactants that can be used in the context of the present invention include those known by persons of ordinary skill in the art (see, e.g., International Cosmetic Ingredient Dictionary and Handbook, 12^{th} Ed. (2008), which is incorporated by reference). Non-limiting examples include: amphiphile, CHAPS detergent, cocamidopropyl betaine, cocamidopropyl hydroxysultaine, hydroxysultaine, lecithin, and sodium lauroamphoacetate. Zwitterionic surfactants such as those described in the specification are commercially available from a wide variety of sources (see, e.g., International Cosmetic Ingredient Dictionary and Handbook, 12^{t1} Ed. (2008), which is incorporated by reference).

### E. Non-Lipid Structuring Agents

The non-lipid structuring agent is used to provide structure to the overall composition. In one aspect, it can help stabilize the composition. Non-limiting examples of non-lipid structuring agents include those known by persons of ordinary skill in the art (see, e.g., International Cosmetic Ingredient Dictionary and Handbook, 12^{th} Ed. (2008), which is incorporated by reference). Acrylates copolymers are polymers formed from at least two monomers selected from acrylic and methacrylic acids, and esters and/or amides of acrylic acid or methacrylic acid. Non-limiting examples of such copolymers that can be used in the context of the present invention include those known by persons of ordinary skill in the art (see, e.g., International Cosmetic Ingredient Dictionary and Handbook, 12^{th} Ed. Vol. 4, pages 4059-4060 (2008), which is incorporated by reference).

### F. Compositions of the Present Invention

It is contemplated that the compositions of the present invention can include a skin moisturizing agent, a cationic guar gum, an anionic surfactant, a zwitterionic surfactant, and a non-lipid structuring agent. Additionally, the compositions can include any number of combinations of additional ingredients described throughout this specification. The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight (w/w) or volume of the total composition. A person of ordinary skill in the art would understand that the concentrations can vary depending on the addition, substitution, and/or subtraction of ingredients in a given composition.

### G. Additional Ingredients

In addition to the skin moisturizing agent, the cationic guar gum, the anionic surfactant, the zwitterionic surfactant, and the non-lipid structuring agent, disclosed throughout this specification, compositions of the present invention can include additional ingredients such as cosmetic ingredients and pharmaceutical active ingredients. Non-limiting examples of these additional ingredients are described in the following subsections.

1. Cosmetic Ingredients

The CTFA International Cosmetic Ingredient Dictionary and Handbook (2004 and 2008) describes a wide variety of non-limiting cosmetic ingredients that can be used in the context of the present invention. Examples of these ingredient classes include: fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraminobenzoic acid (“PABA”) and corresponding PABA derivatives, titanium dioxide, zinc oxide, etc.), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and manitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate). Non-limiting examples of some of these ingredients are provided in the following subsections.

a. UV Absorption Agents

UV absorption agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. Non-limiting examples of physical sunblocks include, kaolin, talc, petrolatum and metal oxides (e.g., titanium dioxide and zinc oxide). Compositions of the present invention can have UVA and UVB absorption properties. The compositions can have an sun protection factor (SPF) of 2, 3, 4, 5, 6, 7, 8, 9, 10, 11, 12, 13, 14, 15, 20, 25, 30, 35, 40, 45, 50, 55, 60, 70, 80, 90 or more, or any integer or derivative therein.

b. Moisturizing Agents

Additional moisturizing agents that can be used include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, alanine, algae extract, aloe barbadensis, aloe-barbadensis extract, aloe barbadensis gel, althea officinalis extract, apricot (prunus armeniaca) kernel oil, arginine, arginine aspartate, arnica montana extract, aspartic acid, avocado (persea gratissima) oil, barrier sphingolipids, butyl alcohol, beeswax, behenyl alcohol, beta-sitosterol, birch (betula alba) bark extract, borage (borago officinalis) extract, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (oenothera biennis) oil, fatty acids, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, maltitol, matricaria (chamomilla recutita) oil, methyl glucose sesquistearate, methylsilanol PCA, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinol palmitate, rice (oryza sativa) bran oil, RNA, rosemary (rosmarinus officinalis) oil, rose oil, safflower (carthamus tinctorius) oil, sage (salvia officinalis) oil, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

c. Antioxidants

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCI, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

d. Structuring Agents

In other non-limiting aspects, the compositions of the present invention can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

e. Emulsifiers

In certain aspects of the present invention, the compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

f. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

g. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

h. Thickening Agents

Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene or trihydroxystearin, or a mixture of both.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

i. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

2. Pharmaceutical Ingredients

Pharmaceutical active agents are also contemplated as being useful with the compositions of the present invention. Non-limiting examples of pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### H. Kits

Kits are also contemplated as being used in certain aspects of the present invention. For instance, compositions of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Moisturizing/Cleansing Composition

A non-limiting example of a composition of the present invention that has the ability to both moisturize and cleanse skin at the same time is described in Tables 1-2.

TABLE 1*

| Ingredient | % Concentration (by weight) |
|---|---|
| Phase A | |
| Water | q.s. |
| Methylparaben | 0.200 |
| EDTA | 0.100 |
| Citric Acid | 0.005 |
| Sodium Chloride | 0.005 |
| Sodium Laureth Sulfate | Approx. 16.2 |
| Acrylate Copolymer | 5.000 |
| Phase B | |
| Mineral Oil (Heavy) | 18.000 |
| Lauric Acid | 3.000 |
| Phase C | |
| Guar Hydroxypropyltrimonium Chloride | 0.300 |
| Glycerin | 2.000 |
| Phase D | |
| Cocamidopropyl Hydroxysultaine | Approx. 5 |
| Peg-120 Methyl Glucose Dioleate | 0.500 |
| Phase E | |
| Triethanolamine (TEA) | 2.500 |
| Phase F | |
| Fragrance** | 2.000 |
| Phase G | |
| DMDM Hydantoin | 0.300 |
| | 100% |

*The Table 1 cleanser formulation was prepared as follows: (1) Weigh out phase A ingredients and mix until uniform; (2) Heat phase A to 75° C.; (3) Heat phase B (premix) to 75° C. and add to phase A and mix until batch looks uniform. Mix for a minimum of 30 minutes; (4) Add phase C (premix) to batch. Mix until uniform and maintain batch at 75° C.; (5) Add phase D and mix until batch is uniform; (6) When batch looks uniform, add phase E and increase mixing speed as batch thickens. Mix for 30 minutes at 75° C. and then switch to sweep mixing. Start cooling batch to 45° C.; (7) when batch reaches 50° C., add phase F and phase G at 45° C. and continue mixing until batch reaches 25° C.
**Any fragrance can be used. Further, the Fragrance can be removed and the composition can be q.s. ed with water.

The Table 1 composition produced a cleansing and skin moisturizing effect when applied to skin and subsequently rinsed with water (data not shown). However, the stability of the formulation was not optimal as evidenced by phase separation over a specified period of time (data not shown).

The Table 2 composition produced a cleansing and skin moisturizing effect when applied to skin and subsequently rinsed with water (data not shown).

TABLE 2*

| Ingredient | % Concentration (by weight) |
|---|---|
| Phase A | |
| Water | q.s. |
| Methylparaben | 0.200 |
| EDTA | 0.100 |
| Citric Acid | 0.005 |
| Sodium Chloride | 0.005 |
| Sodium Laureth Sulfate | Approx. 16.2 |
| Acrylate Copolymer | 5.500 |
| Phase B | |
| Mineral Oil (Heavy) | 18.000 |
| Lauric Acid | 3.500 |
| Phase C | |
| Guar Hydroxypropyltrimonium Chloride | 0.300 |
| Glycerin | 2.000 |
| Phase D | |
| Cocamidopropyl Hydroxysultaine | Approx. 5 |
| Peg-150 Pentaerythrityl Tetrastearate | 0.400 |
| Phase E | |
| Triethanolamine (TEA) | 2.800 |
| Phase F | |
| Fragrance** | 2.000 |
| Phase G | |
| DMDM Hydantoin | 0.300 |
| | 100% |

*The Table 2 cleanser formulation was prepared as follows: (1) Weigh out phase A ingredients and mix until uniform; (2) Heat phase A to 75° C.; (3) Heat phase B (premix) to 75° C. and add to phase A and mix until batch looks uniform. Mix for a minimum of 30 minutes; (4) Add phase C (premix) to batch. Mix until uniform and maintain batch at 75° C.; (5) Add phase D and mix until batch is uniform; (6) When batch looks uniform, add phase E and increase mixing speed as batch thickens. Mix for 30 minutes at 75° C. and then switch to sweep mixing. Start cooling batch to 45° C.; (7) when batch reaches 50° C., add phase F and phase G at 45° C. and continue mixing until batch reaches 25° C.
**Any fragrance can be used. Further, the Fragrance can be removed and the composition can be q.s. ed with water.

Surprisingly, the stability of the Table 2 composition was far greater when compared with the Table 1 composition to the point that the Table 2 composition had an acceptable shelf-life, which allows for longer periods of storage and use time for the end user. The stability improvement of the Table 2 composition can be attributed to the following changes to the Table 1 composition: the amount of acrylate copolymer was increased to 5.5% by weight; lauric acid was increased to 3.5% by weight; PEG-120 Methyl Glucose Dioleate was removed and replaced with 0.4% by weight of PEG-150 Pentaerythrityl tetrastearate; and the amount of TEA was increased to 2.8% by weight. Water was decreased in the Table 2 formulation due to these changes.

In an additional experiment, the Table 2 formulation was further revised by replacing mineral oil with petrolatum, adding small amounts of shea butter and tocopheryl acetate (less than 1% wt %), and q.s.ing the water as needed. This revised Table 2 formulation had positive stability and cleansing/moisturizing effects while also producing a composition that had a desirable tactile feel.

In addition, the skin moisture/hydration can be measured by using impedance measurements with the Nova Dermal Phase Meter. The impedance meter measures changes in skin moisture content. The outer layer of the skin has distinct electrical properties. When skin is dry it conducts electricity very poorly. As it becomes more hydrated increasing conductivity results. Consequently, changes in skin impedance (related to conductivity) can be used to assess changes in skin hydration. The unit can be calibrated according to instrument instructions for each testing day. A notation of temperature and relative humidity can also be made. Subjects can be evaluated as follows: prior to measurement they can equilibrate in a room with defined humidity (e.g., 30-50%) and temperature (e.g., 68-72 C). Three separate impedance readings can be taken on each side of the face, recorded, and averaged. The T5 setting can be used on the impedance meter which averages the impedance values of every five seconds application to the face. Changes can be reported with statistical variance and significance.

Skin dryness can be evaluated with clinical grading techniques. For example, clinical grading of skin dryness can be determined by a five point standard Kligman Scale: (O) skin is soft and moist; (1) skin appears normal with no visible dryness; (2) skin feels slightly dry to the touch with no visible flaking; (3) skin feels dry, tough, and has a whitish appearance with some scaling; and (4) skin feels very dry, rough, and has a whitish appearance with scaling. Evaluations can be made independently by two clinicians and averaged.

Skin softness/suppleness can be evaluated using the Gas Bearing Electrodynamometer, an instrument that measures the stress/strain properties of the skin. The viscoelastic properties of skin correlate with skin moisturization. Measurements can be obtained on the predetermined site on the cheek area by attaching the probe to the skin surface with double-stick tape. A force of approximately 3.5 gm can be applied parallel to the skin surface and the skin displacement is accurately measured. Skin suppleness can then be calculated and is expressed as DSR (Dynamic Spring Rate in gm/mm).

All of the compositions and/or methods disclosed and claimed in this specification can be made and executed without undue experimentation in light of the present disclosure. While the compositions and methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A liquid skin cleansing and moisturizing composition comprising:
(a) a hydrophobic phase comprising petrolatum in an amount of at least 50% by weight of the hydrophobic phase;
(b) a hydrophilic phase comprising:
(i) water;
(ii) a cationic guar gum; and
(iii) a non-lipid structuring agent; and
(c) a combination of anionic and zwitterionic surfactants,
wherein the total amount of water in the composition is less than 50% by weight of the composition,
wherein the total amount of surfactants within the composition is between 15 and 25% by weight of the composition,
wherein the total amount of the non-lipid structuring agent within the composition is between 3 to 7% by weight of the composition,
wherein the composition is in liquid form and has a viscosity range between 15,000 to 25,000 cps, as measured on a Brookfield Viscometer using a TC spindle at 2.5 rpm at 25° C., and
wherein the composition does not include a crystalline hydroxy-containing stabilizer and a tri-hydroxystearin.

2. The composition of claim 1, wherein the hydrophobic phase consists of petrolatum, shea butter extract, and tocopheryl acetate.

3. The composition of claim 1, wherein the composition does not include ammonium laureth sulfate, ammonium lauryl sulfate, sodium lauryoamphoacetate, trihydroxystearin, sodium benzoate, niacinamide, PEG-14, retinyl palmitate, glycine soja oil, sunflower seed oil, cocamidopropyl betaine, cocamide MEA, polybutene, polyisobutene, isostearic acid, PEG-5 cocamide, acrylates/beheneth-25 methacrylate copolymer, PEG-30 dipolyhydroxystearate, tetrasodium EDTA, etidronic acid, iodopropynyl butylcarbamate, titanium dioxide, hydrogenated polyisobutene, sodium lauryoyl sarcosinate, sodium hydroxypropyl starch phosphate, lauryl alcohol, propylene glycol, and cyamopsis tetragonoloba (guar) gum.

4. The composition of claim 1, wherein the a hydrophobic phase comprises petrolatum in an amount of at least 85% by weight of the hydrophobic phase.

5. The composition of claim 1, wherein the non-lipid structuring agent is an acrylates copolymer.

6. The composition of claim 1, wherein the anionic surfactant is sodium laureth sulfate, the zwitterionic surfactant is cocamidopropyl hydroxysultaine, and the non-lipid structuring agent is an acrylates copolymer.

7. A liquid skin cleansing and moisturizing composition comprising:
(a) 15 to 20% by weight of the composition of petrolatum;
(b) 0.1 to 1% w/w of guar hydroxypropyltrimonium chloride;
(c) 15 to 20% w/w of an anionic surfactant;
(d) 3 to 7% w/w of a zwitterionic surfactant; and
(e) 3 to 7% w/w of a non-lipid structuring agent.

8. The composition of claim 7, wherein the anionic surfactant is sodium laureth sulfate, the zwitterionic surfactant is cocamidopropyl hydroxysultaine, and the non-lipid structuring agent is an acrylates copolymer.

9. The composition of claim 8, further comprising:
(f) water;
(g) EDTA;
(h) citric acid;
(i) sodium chloride;
(j) lauric acid;
(k) glycerin;
(l) PEG-120 methyl glucose dioleate or PEG-150 Pentaerythrityl tetrastearate;
(m) triethanolamine;
(n) DMDM hydantoin;
(o) a fragrance; and
(p) a preservative.

10. The composition of claim 9, comprising:
(f) less than 50% w/w of water;
(g) 0.1 to 0.5% w/w of EDTA;
(h) 0.001 to 0.01% w/w of citric acid;
(i) 0.001 to 0.01% w/w of sodium chloride;
(j) 1 to 5% w/w of lauric acid;
(k) 1 to 5% w/w of glycerin;
(l) 0.1 to 1% w/w of PEG-120 methyl glucose dioleate or PEG-150 Pentaerythrityl tetrastearate;
(m) 1 to 5% w/w of triethanolamine;
(n) 0.1 to 1% w/w of DMDM hydantoin;
(o) 1 to 5% w/w of a fragrance; and
(p) 0.1 to 1% w/w of a preservative.

11. The composition of claim 10 further comprising tocophyeryl acetate and shea butter extract.

12. The composition of claim 11, wherein the composition consists of the claimed ingredients.

13. The composition of claim 11, wherein the composition consists essentially of the claimed ingredients.

14. The composition of claim 11, wherein the composition does not include ammonium laureth sulfate, ammonium lauryl sulfate, sodium lauryoamphoacetate, trihydroxystearin, sodium benzoate, niacinamide, PEG-14, retinyl palmitate, glycine soja oil, sunflower seed oil, cocamidopropyl betaine, cocamide MEA, polybutene, polyisobutene, isostearic acid, PEG-5 cocamide, acrylates/beheneth-25 methacrylate copolymer, PEG-30 dipolyhydroxystearate, tetrasodium EDTA, etidronic acid, iodopropynyl butylcarbamate, titanium dioxide, hydrogenated polyisobutene, sodium lauryoyl sarcosinate, sodium hydroxypropyl starch phosphate, lauryl alcohol, propylene glycol, and cyamopsis tetragonoloba (guar) gum.

15. The composition of claim 11, wherein the composition does not include a lamellar phase and does not include a crystalline hydroxy-containing stabilizer and a tri-hydroxystearin.

16. A method of cleansing and moisturizing skin comprising:
(a) applying the composition of claim 1 to skin; and
(b) rinsing the skin having the composition applied thereto with water within 5 minutes after the composition has been applied to the skin.
